(19) United States
(12) Patent Application Publication
(10) Pub. No.: US 2009/0155345 A1
(43) Pub. Date: Jun. 18, 2009

(54) METHODS FOR AFFECTING LIPOSOME COMPOSITION ULTRASOUND IRRADIATION

(75) Inventors: Yechezkel Barenholz, Jerusalem (IL); Avi Schroeder, Dn Sde Gat (IL); Joseph Kost, Omer (IL)

Correspondence Address: BROOKS KUSHMAN P.C., 1000 TOWN CENTER, TWENTY-SECOND FLOOR, SOUTHFIELD, MI, 48075, US

(73) Assignees: BEN GURION UNIVERSITY OF THE NEGEV RESEARCH AND DEVELOPMENT AUTHORITY, BEER SHEVA (IL); YISSUM RESEARCH DEVELOPMENT COMPANY OF THE HEBREW UNIVERSITY OF JERUSALEM, JERUSALEM (IL); HI TECH PARK EDMOND CAMPUS GIVAT RAM, JERUSALEM (IL)

(21) Appl. No.: 12/096,652

(22) PCT Filed: Dec. 7, 2006
(86) PCT No.: PCT/IL06/01404

§ 371 Date: Oct. 16, 2008

Related U.S. Application Data
(60) Provisional application No. 60/748,149, filed on Dec. 8, 2005.

(51) Int. Cl. A61K 9/127; B01J 13/02

(52) U.S. Cl. 424/450; 264/4.1

(57) ABSTRACT

The present invention provides methods for loading of agents and substances into per-formed liposomes, preferably a suspension of pre-formed liposomes as well as to methods for the controlled quantum (step-wise) release of agents and substances from liposomes. One of the principle features of the methods of the invention is to expose the liposomes to ultrasound irradiation having predefined parameters, resulting in an increase in permeability of the liposomes, thereby permitting, respectively, the loading and/or release of agents and substances into and/or from the liposomes.

### FIELD OF THE INVENTION

This invention relates to liposome technology and in particular to therapeutic applications of liposomes in combination with low frequency ultrasound (LFUS).

### LIST OF PRIOR ART

The following is a list of prior art, which is considered to be pertinent for describing the state of the art in the field of the invention.
1. Gao, Z. G.; Fain, H. D.; Rapoport, N.; Controlled and targeted chemotherapy by micellar encapsulated drug and ultrasound. J. Control. Release, 2005. 102(1): 203-222
2. Pitt, W. G.; Husseini, G. A.; Staples, B. J.; Ultrasonic drug delivery—a general review. Epert Opin. Drug Deliv. 2004, 1(1): 37-56.
3. Lavon, I.; Kost, J.; Ultrasound and transdermal drug delivery. Drug Discovery Today. 2004, 9(15), 670-676.
4. Tang, H.; Wang, C. C. J.; Blankschtein, D.; Langer, R., An Investigation of the role of cavitation in low-frequency ultrasound-mediated transdermal drug transport. Pharm. Res. 2002, 19 (8), 1160-1169.
5. Anwerl, K.; Kao, G.; Proctor, B.; Anscombe, I.; Florack, V.; Earls, R.; Wilson, E.; McCreery, T.; Unger, E.; Rolland, A.; Sullivan, S. M.; Ultrasound enhancement of cationic lipid mediated gene transfer to primary tumors following systemic administration. Gene Therapy, 2000, 7, 1833-1839.
6. Duvshani-Eshet, M.; Baruch, L.; Kesselman, E.; Shimoni, E.; Machluf, M.; Therapeutic ultrasound mediated DNA to cell and nucleus: bioeffects revealed by confocal and atomic force microscopy. Gene Therapy, 2006. 13, 163-172.
7. Sundaram, J., Mellein B. R., and Mitragotri S., An experimental and theoretical analysis of ultrasound-induced permeabilization of cell membranes. Biophysical J. 2003 84, 3087-3101.
8. Barenholz, Y.; Cevc, G., Structure and properties of membranes in Physical Chemistry of Biological Surfaces. Marcel Dekker: New York, 2000; p 171-241.
9. Lin, H.-Y.; Thomas, J. L., PEG-Lipids and Oligo(ethylene glycol) Surfactants Enhance the Ultrasonic Permeabilizability of Liposomes. Langmuir 2003, 19 (4), 1098-1105.
10. Lin, H.-Y.; Thomas, J. L., Factors Affecting Responsivity of Unilamellar Liposomes to 20 kHz Ultrasound. Langmuir 2004, 20 (15), 6100-6106.
11. Cohen-Levi, D.; Kost, J.; Barenholz, Y. Ultrasound for targeted delivery of cytotoxic drugs from liposomes, MSc Thesis. Ben Gurion University, Beer Sheva, Israel, 2000.

### BACKGROUND OF THE INVENTION

Ultrasound and in particular low frequency ultrasound (LFUS) has been shown to enhance the permeability of biological membranes for drug and gene delivery.¹⁻⁷ The fact that the bilayer structure, as well as many physicochemical properties of the liposome membrane are similar to those of biological membranes,⁸ led us to determine whether LFUS can increase the permeability of liposomes to release the entrapped drug in a controlled manner.

Earlier studies have shown that liposomes release calcein from their intraliposomal aqueous phase in response to 20 kHz ultrasonic irradiation.^{9,10} Another study showed that 20-kHz ultrasonic irradiation is more efficient in releasing doxorubicin from liposomes than high-frequency ultrasound (1 and 3 MHz)¹¹. Further, treating BALB/c mice inoculated with human colon cancer tumors with Doxil and later, after the liposomes have accumulated in the tumor site, exposing the tumors to LFUS was shown to reduce tumor size.

### SUMMARY OF THE INVENTION

The present invention is based, inter alia, on the following two findings:
Ultrasound (US) irradiation of pre-formed liposomes facilitates loading of various materials into the lipid membrane and/or into the liposomal aqueous core of the liposomes.
By the use of US irradiation it is possible to control and quantify the release of various materials loaded into a liposome (either in the liposome's membrane or in the liposomal aqueous core). Specifically, it has been shown that by applying a series of US irradiation sessions it is possible to dictate a quantum release of material loaded in liposomes so that the compound is released from the liposome step wise in a controlled manner.

Thus, in accordance with a first aspect of the invention (herein “the loading aspect of the invention”) there is provided a method for loading an agent into a pre-formed liposomes comprising:
(a) contacting said pre-formed liposomes with said agent; and
(b) subjecting the pre-formed liposomes to ultrasound (US) irradiation;
wherein said contacting of the preformed liposomes with said agent is before or after said US irradiation; and said US irradiation comprises parameters being effective to increase permeability of said liposomes and thereby permit loading of said agent into said liposomes.

In accordance the loading aspect of the invention there is also provided a method for reducing the amount of a substance in a fluid medium or tissue comprising:
(a) contacting the fluid medium or tissue with pre-formed liposomes;
(b) subjecting the pre-formed liposomes to US irradiation;
wherein said contact of the fluid medium or tissue with the pre-formed liposomes is before or after said irradiation; and said US irradiation comprises parameters being effective to increase permeability of said liposomes, permitting loading of said substance into said liposomes thereby reducing the amount of the substance in the medium or tissue.

Further provided in accordance with the loading aspect of the invention, is a method for reducing the level of a substance in a subject's body, the method comprises:
(a) administering to said subject an amount of pre-formed liposomes in a manner permitting contact between said pre-liposomes and said substance;
(b) subjecting the pre-formed liposomes to US irradiation;
wherein said irradiation of the pre-formed liposomes is before or after administration of the liposomes to said subject's body; and said US irradiation comprises parameters being effective to increase permeability of said liposomes and thereby loading of said substance into said liposomes, which results in reduction of the level of the substance in said subject's body.

Further, in accordance with the loading aspect of the invention there is provided a kit comprising:
(a) a composition of pre-formed liposomes;
(b) instructions for subjecting said composition of pre-formed liposomes to US irradiation, said instructions identifying irradiation parameters for said US irradiation which induce an increase in permeability of the pre-formed liposomes, such that when the irradiated liposomes are brought into contact with a substance, at least a portion of said substance is loaded into said liposomes.

Finally, in accordance with the loading aspect of the invention there is provided the use of pre-formed liposomes for the preparation of a pharmaceutical composition for removing a substance from a subject's body, said composition being intended for use in combination with exposing said pre-formed liposomes to US irradiation when said composition is within said subject's body.

In accordance with a second aspect of the invention (herein “the release aspect of the invention”) there is provided a method for the quantum release from liposomes of an agent stably loaded into said liposomes, the method comprises subjecting said liposomes to a series of two or more US irradiation sessions, said US irradiation comprises parameters being effective to increase permeability of said liposomes thereby permitting release of an amount of said agent from said liposomes.

Further, in accordance with the release aspect of the invention there is provided a kit comprising:
(a) a composition of liposomes loaded with an agent;
(b) instructions for applying a series of two or more US irradiation sessions on a subject's body following administration of said composition of liposomes to said subject, said instructions comprising an index identifying irradiation parameters for each irradiation session and the amount of agent released from said liposomes during an identified irradiation session.

In accordance with another embodiment within the release aspect of the invention there is provided a kit comprising:
(a) a composition of liposomes loaded with an agent;
(b) instructions for applying a series of two or more US irradiation sessions on a subject's body following administration of said composition of liposomes to said subject, said instructions comprise an index of treatment protocols corresponding to patient and disease-related parameters, the treatment protocols defining irradiation parameters.

Finally, in accordance with the release aspect of the invention there is provided the use of liposomes loaded with an agent for the preparation of a pharmaceutical composition for quantum release of the agent from the liposomes as a result of exposure said liposomes to US irradiation.

It is noted that in the context of the present invention the loading and releasing aspects of the invention may be combined. For example, liposomes loaded with a substance, may be effective to release the substance upon exposures to US irradiations while simultaneously or thereafter and during the same or following US irradiations be effective to load another substance present in the surrounding medium or tissue.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a graph showing the effect of low frequency US (LFUS) irradiation time on liposome uptake of a membrane-impermeable fluorescent probe, pyranine, from the extraliposomal medium;

FIG. 2 is a bar graph showing Zeta Potential of liposomes incubated with a cationic lipid DOTAP, an anionic lipid DMPG or the control (no lipid) following exposure to LFUS or without any exposure to LFUS.

FIG. 3 is a graph showing the effect of ultrasound amplitude on Methylprednisolone hemisuccinate sodium salt (MPS) release from liposomes.

FIG. 4 is a graph showing the effect of US on release of three different liposomal (SSL) drug formulations: (-▪-) MPS, (-▴-) doxorubicin (Doxil), (-♦-) cisplatin (Stealth cisplatin), and (-□-) SSL with a high intraliposomal/low extraliposomal calcium acetate gradient.

FIG. 5 is a graph showing LFUS-triggered MPS release from liposomes following continuous (-♦-) or pulsed (-□-) irradiation modes.

FIGS. 6a-6c are cryo-transmission electron microscopy images of liposomes before remote loading of MPS (FIG. 6a); liposomes after remote loading of MPS (FIG. 6b); liposomes remote loaded with MPS after being exposed to LFUS (20 kHz, 120 s, 3.3 W/cm²) (FIG. 6c).

FIG. 7 is a graph showing the effect of LFUS irradiation time on liposomal MPS dispersions: turbidity (left axis, -□-), and dynamic light scattering (DLS) signal intensity (right axis, -▴-).

FIG. 8 is a graph showing the effect of LFUS irradiation time on liposomal MPS mean size, as assessed by dynamic light scattering at 90°.

FIG. 9 is a graph showing the concentrations of total (liposomal plus non-liposomal) phospholipid (-□-) and of liposomal phospholipid (-♦-) in LFUS-irradiated liposomal dispersions.

FIG. 10 is an image showing the effect of LFUS on lipid chemical stability, based on TLC analysis of extracted lipids.

FIG. 11 is a graph showing cytotoxicity of cisplatin released from Stealth-cisplatin liposomes by LFUS to C26 murine colon adenocarcinoma cells in culture.

### DETAILED DESCRIPTION OF EXEMPLARY EMBODIMENTS

In the past years interest has been made in using ultrasound to increase permeability of cellular membranes for DNA transfection and for drug delivery [Lin, H.-Y.; et al. Langmuir 2004, 20 (15), 6100-6106]. The different bilayer membranes exhibited different responsivity to ultrasound [Lin, H.-Y.; et al. Langmuir 2003, 19 (4), 1098-1105].

Based on the results presented herein, the inventors has thus envisaged that ultrasound-induced permeability of liposomes is an important tool for loading of various materials into the liposomes as well as for the controlled release of various materials from liposomes.

Without wishing to be bound by theory it is believed that the mechanism of release from, or loading into liposomes in accordance with the invention is suggested to be associated with morphological changes such as, without being bound by theory, the transient formation of pore-like defects in the liposome membrane through which the material may be released or introduced into the liposomes. These defects are most likely caused by US-induced cavitation occurring near the liposome membrane in the extraliposomal medium, and/or by small cavitation nuclei in the intraliposomal aqueous compartment. The pore-like defects in the membrane typically reseal once US irradiation has stopped (unless the liposomes have been designed otherwise as will be discussed below).

The following examples show that exposure to US modified neither the chemical properties of the irradiated liposomal drugs or lipids, nor the biological activity of these drugs.

Thus, in accordance with a first of its aspects, the present invention provides methods for loading of agents and substances into per-formed liposomes, preferably a suspension of pre-formed liposomes. This aspect of the invention is referred to herein as “the loading aspect of the invention”.

In accordance with a second of its aspects, the present invention provides methods for controlled release of agents and substances from liposomes. This aspect of the invention is referred to herein as “tie release aspect of the invention”.

The liposomes, in accordance with both aspects of the invention, are designed to have low permeability.

“Permeability” is generally defined by the amount of a specific material that permeates or leaks per unit of area and unit of time. In the context of the present invention, “permeability” denotes the capability of a lipid bilayer forming the liposome's membrane to spontaneously or passively transfer (without manipulations such as irradiation or heating) over time a substance, e.g. a drag or other agent, from one side of the liposome membrane (e.g. from the inter-liposomal aqueous medium, also referred to as extra-liposome medium) to the other side of the membrane (e.g., to the intra-liposomal core). Permeability of the liposomes may be determined by methods known in the art to measure cell and liposome permeability. For example, leakage of an agent can be measured by separating the liposomes from any material which has leaked out, using methods such as gel permeation chromatography, dialysis, ultra-filtration or the like, and assaying in a known manner for any leaked material (see also in this connection sections relating to permeability in: Liposomes: A Practical Approach. V Weissig & V Torchilin (eds), 2^{nd} edition; New, R. C. C., Liposomes: A Practical Approach, Oxford 1^{st} edition).

Thus, in the context of the present invention, the term “low permeability” is defined as the capability of the liposomes to spontaneously release no more than 10% of a material a priori loaded into a liposome during a storage period of at least one month or alternatively, the capability to spontaneously load no more than 0.1% of a material dissolved or dispersed in the medium surrounding pre-formed liposomes during a storage period of at least one month.

A variety of factors have been shown to influence liposome permeability. Permeability is enhanced near and at the phase transition temperature; it is reduced by the incorporation of sterols such as cholesterol. Detergents and other amphiphiles with large head groups also increase permeability, at concentrations well below that required for solubilization. Thus, distinctively different permeabilities may be achieved by using different components within the bilayer of the two liposome populations. In fact, lipid composition is the main factor in the determination of liposome permeability, and as indicated above this correlates with a lipid's (when using one lipid to form the liposome) Tₘ. Cholesterol will slow down leakage (i.e. reduce permeability) when all the lipids in the bilayer are in the liquid ordered (LO) phase (in many cases with PCs it is obtained with an amount of cholesterol being equal or above 33 mole %). For liposomes composed of a mixture of liposome-forming lipids the parameters may be complex, as appreciated and known by those versed in the art.

Temperature also affects permeability. The permeability of liposomes in the liquid disordered (LD) phase will be higher than the permeability of liposomes in the solid ordered (SO) or liquid ordered (LO) phases.

Size of liposomes may have some effect as the membrane permeability, as large liposomes (e.g., 100 nm and above) have less curvature than the membranes of liposomes smaller than 100 nm. Another difference between large and small liposomes is in the surface area/volume ratio which, for large liposomes, is smaller than for small liposome; and therefore more material will leak from the small liposomes as compared to larger liposomes. However, these differences may be considered as mild.

Finally, as appreciated, for leakage or permeation through a membrane of multilamellar vesicles (MLV) it is required that the material cross more than one bilayer to become free (or entrapped). MLV may therefore be regarded as less permeable than unilamellar liposomes. The same understanding should apply to multivesicular vesicles. However, also in this case, the differences in permeability between multi- and univesicular vesicles are mild.

In accordance with one embodiment, the permeability of the liposomes is achieved by using lipids having a defined gel to liquid crystalline phase transition temperatures (Tₘ).

A thermotropic phase transition from gel (i.e. solid or solid ordered, SO) to liquid crystalline (i.e. fluid or liquid disordered, LD) or from liquid crystalline to gel phase undergone by lipids and liposomes is known to affect the free volume and degree of rigidity of the lipid bilayer of the liposome. When in LD phase, the lipids in both leaflets forming the bilayer are “loosely” aligned according to their hydrophilic and lipophilic regions. This packing enables a large level of “free volume” which facilitates diffusion across the liposome membrane. Below the range of main transition (i.e. when in the SO phase), the lipid molecules are more closely packed, and the lipid bilayers has much less free volume, and therefore permeability is reduced to a large extent, and may be eliminated entirely.

It is noted that at the temperature range in which LD and SO phases co-exist there are interface regions in which packing is disturbed and there are packing defects, as the two phases do not fit to each other. At this range permeability is usually the highest.

As indicated above, permeability may also be designed by adding to the liposome composition membrane active sterols (as briefly discussed above). For example, in liposomes composed mainly of PCs and/or sphingomyelins (or any other liposome forming lipid, excluding those having polyunsaturated acyl chains) and having cholesterol in an amount between 25 to 50 mole %, all the bilayer is in the LO phase. As a result, permeability is reduced compared to liposomes in the LD phase. However there is no risk of going through the main transition as this is abolished by the high mole % of cholesterol (or other membrane active sterol). When comparing permeability of liposomes of different liposome-forming lipids with the same level of membrane active sterol (like cholesterol), permeability will be determined by each liposome-forming lipid's Tₘ. For example, permeability of HSPC/cholesterol (Tₘ of HSPC is 52° C.) is lower than that of DPPC/Cholesterol (Tₘ of DPPC 41.4° C.) or that of DMPC/Cholesterol (Tₘ of DMPC is 23.5° C.).

It is also worth noting that permeability of a membrane to a material may also depend on the characteristics of the specific material and in particular, the material's octanol to aqueous phase partition coefficient (Kp). For example, doxorubicin has a low Kp and bupivacaine a much higher Kp. This explains the differences in their leakage rate from the same liposomes or from liposomes of similar composition. For this reason, bupisomes (bupivacaine-loaded liposomes) leak during storage at 4° C. while Doxil (doxorubicin-loaded liposomes) do not [see also Haran, G.; et al. Biochim. Biophys. Acta, Biomembranes 1993, 1151 (2), 201-215]].

Lipids having a relatively high Tₘ may be referred to as “rigid” lipids, typically those having saturated, long acyl chains, while lipids with a relatively low Tₘ may be referred to as “fluid” lipids. Fluidity or rigidity of the liposome may be determined by selecting lipids with pre-determined fluidity/rigidity for use as the liposome-forming lipids. The selection of the lipids with a specific Tm will depend on the temperature in which the method is to be conducted. For example, when the temperature of the environment is ambient temperature, the lipid(s) forming the liposomes would be such that the phase transition temperature, Tₘ is above ambient temperature, e.g. above 25° C. Further, as an example, when the method of the invention is to be conducted at 4° C., the lipid(s) forming the liposomes are selected such that the Tm is above the same. In accordance with one preferred embodiment, the Tₘ of the lipids forming the liposomes is preferably equal to or above 40° C.

A non limiting example of lipids forming the liposomes and having a Tₘ above 40° C. comprises phosphatidylcholine (PC) and derivatives thereof having two acyl (or alkyl) chains with 16 or more carbon atoms. Some preferred examples of PC derivatives which form the basis for the low permeable liposomes in the context of the invention include, without being limited thereto, hydrogenated soy PC(HSPC) having a Tₘ of 52° C., Dipalmitoylphosphatidylcholine (DPPC), having a Tₘ of 41.3° C., N-palmitoyl sphingomyelin having a Tₘ of 41.2° C., distearylphosphatidylcholine (DSPC) having a Tm of 55° C.], N-stearoyl sphingomyelin having a Tₘ of 48° C., distearyolphosphatidylglycerol (DSPG) having a Tₘ of 55° C.], and distearyphosphatidylserine (DSPS) having a Tₘ of 68° C. All these Tₘ data are from http://www.avantilipids.com/PhaseTransitionTemperaturesGlycerophospholipids.html Phase Transition Temperatures or from http://www.lipidat.chemistry.ohio-state.edu/home.stm, as known to those versed in the art. Those versed in the art will know how to select a lipid with a Tₘ either equal or above 40° C. [see also Barenholz, Y., Liposome application: problems and prospects. Curr. Opin. Colloid Interface Sci. 6, 66-77 (2001); Barenholz, Y. and Cevc, G., Structure and properties of membranes. In Physical Chemistry of Biological Surfaces (Baszkin, A. and Norde, W., eds.), Marcel Dekker, NY (2000) pp. 171-241].

In addition to liposome-forming lipids (like PCs and sphingomyelins), membrane active sterols (e.g. cholesterol) and/or phosphatidylethanolamines may be included in the liposomal formulation in order to decrease a membrane's free volume and thereby permeability and leakage of material loaded therein.

Thus, in accordance with another embodiment, the liposomes may comprise cholesterol. Independently, the lipid/cholesterol mole/mole ratio of the liposomes in the liposome populations may be in the range of between about 75:25 and about 50:50. A more specific mole/mole ratio is about 60:40.

The liposome may include other constituents. For example, charge-inducing lipids, such as phosphatidylglycerol, may also be incorporated into the liposome bilayer to decrease vesicle-vesicle fusion, and to increase interaction with cells. Buffers at a pH suitable to make the liposome surface's pH close to neutral can decrease hydrolysis. Addition of an antioxidant, such as vitamin E, or chelating agents, such as Desferal or DTPA, may be used.

The liposonies are formed by the use of liposome forming lipids. In the context of the present invention the term liposome-forming lipids denotes those lipids having a glycerol backbone wherein at least one, preferably two, of the hydroxyl groups at the head group is substituted by one or more of an acyl, an alkyl or alkenyl chain, a phosphate group, preferably an acyl chain (to form an acyl or diacyl derivative), a combination of any of the above, and/or derivatives of same, and may contain a chemically reactive group (such as an amine, acid, ester, aldehyde or alcohol) at the headgroup, thereby providing a polar head group. Sphingolipids, and especially sphingomyelins, are a good alternative to glycerophospholipids.

Typically, a substituting chain, e.g. the acyl, alkyl or alkenyl chain, is between about 14 to about 24 carbon atoms in length, and has varying degrees of saturation, thus resulting in fully, partially or non-hydrogenated (liposome-forming) lipids. Further, the lipid may be of a natural source, semi-synthetic or a fully synthetic lipid, and may be neutral, negatively or positively charged. There are a variety of synthetic vesicle-forming lipids and naturally-occurring vesicle-forming lipids, including the phospholipids, such as phosphatidylcholine (PC), phosphatidylinositol (PI), phosphatidylglycerol (PG), dimyristoyl phosphatidylglycerol (DMPG); egg yolk phosphatidylcholine (EPC), 1-palmitoyl-2-oleoylphosphatidyl choline (POPC), distearoylphosphatidylcholine (DSPC), dimyristoyl phosphatidylcholine (DMPC); phosphatidic acid (PA), phosphatidylserine (PS); 1-palmitoyl-2-oleoylphosphatidyl choline (POPC), and the sphingophospholipids such as sphingomyelins (SM) having 12- to 24-carbon atom acyl or alkyl chains. The above-described lipids and phospholipids whose hydrocarbon chain (acyl/alkyl/alkenyl chains) have varying degrees of saturation can be obtained commercially or prepared according to published methods. Other suitable lipids include in the liposomes are glyceroglycolipids and sphingoglycolipids and sterols (such as cholesterol or plant sterol).

Cationic lipids (mono- and polycationic) are also suitable for use in the liposomes of the invention, where the cationic lipid can be included as a minor component of the lipid composition or as a major or sole component. Such cationic lipids typically have a lipophilic moiety, such as a sterol, an acyl or diacyl chain, and where the lipid has an overall net positive charge. Preferably, the head group of the lipid carries the positive charge. Monocationic lipids may include, for example, 1,2-dimyristoyl-3-trimethylammonium propane (DMTAP); 1,2-dioleyloxy-3-(trimethylamino) propane (DOTAP); N-[1-(2,3-ditetradecyloxy)propyl]-N,N-dimethyl-N-hydroxyethylammonium bromide (DMRIE); N-[1-(2,3-dioleyloxy)propyl]-N,N-dimethyl-N-hydroxy ethyl ammonium bromide (DORIE); N-[1-(2,3-dioleyloxy) propyl]-N,N,N-trimethylammonium chloride (DOTMA); 3β[N—(N′,N′-dimethylaminoethane)carbamoyl]cholesterol (DC-Chol); and dimethyl-dioctadecylammonium (DDAB).

Examples of polycationic lipids may include a lipophilic moiety similar to those described for monocationic lipids, to which the polycationic moiety is attached. Exemplary polycationic moieties include spermine or spermidine (as exemplified by DOSPA and DOSPER), or a peptide, such as polylysine or other polyamine lipids. For example, the neutral lipid (DOPE) can be derivatized with polylysine to form a cationic lipid. Polycationic lipids include, without being limited thereto, N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA), and ceramide carbamoyl spermine (CCS).

Further, the liposomes may also include a lipid derivatized with a hydrophilic polymer to form new entities known by the term lipopolymers. Lipopolymers preferably comprise lipids modified at their head group with a polymer having a molecular weight equal to or above 750 Da. The head group may be polar or apolar; however, it is preferably a polar head group to which a large (>750 Da), highly hydrated (at least 60 molecules of water per head group), flexible polymer is attached. The attachment of the hydrophilic polymer head group to the lipid region may be a covalent or non-covalent attachment; however, it is preferably via the formation of a covalent bond (optionally via a linker). The outermost surface coating of hydrophilic polymer chains is effective to provide a liposome with a long blood circulation lifetime in vivo. The lipopolymer may be introduced into the liposome in two different ways either by: (a) adding the lipopolymer to a lipid mixture, thereby forming the liposome, where the lipopolymer will be incorporated and exposed at the inner and outer leaflets of the liposome bilayer [Uster P. S. et al. FEBBS Letters 386:243 (1996)]; or (b) first preparing the liposome and then incorporating the lipopolymers into the external leaflet of the pre-formed liposome either by incubation at a temperature above the Tₘ of the lipopolymer and liposome-forming lipids, or by short-term exposure to microwave irradiation.

Liposomes may be composed of liposome-forming lipids and lipids such as phosphatidylethanolamines (which are not liposome forming lipids) and derivatization of such lipids with hydrophilic polymers the latter forming lipopolymers which in most cases are not liposomes-forming lipids. Examples have been described in Tirosh et al. [Tirosh et al., Biopys. J., 74(3):1371-1379, (1998)] and in U.S. Pat. Nos. 5,013,556; 5,395,619; 5,817,856; 6,043,094; and 6,165,501; incorporated herein by reference; and in WO 98/07409. The lipopolymers may be non-ionic lipopolymers (also referred to at times as neutral lipopolymers or uncharged lipopolymers) or lipopolymers having a net negative or a net positive charge.

There are numerous polymers which may be attached to lipids. Polymers typically used as lipid modifiers include, without being limited thereto: polyethylene glycol (PEG), polysialic acid, polylactic acid (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

While the lipids derivatized into lipopolymers may be neutral, negatively charged, or positively charged, i.e. there is no restriction regarding a specific (or no) charge, the most commonly used and commercially available lipids derivatized into lipopolymers are those based on phosphatidyl ethanolamine (PE), usually, distearylphosphatidylethanolamine (DSPE).

A specific family of lipopolymers which may be employed by the invention include monomethylated PEG attached to DSPE (with different lengths of PEG chains, the methylated PEG referred to herein by the abbreviation PEG) in which the PEG polymer is linked to the lipid via a carbamate linkage resulting in a negatively charged lipopolymer. Other lipopolymer are the neutral methyl polyethyleneglycol distearoylglycerol (mPEG-DSG) and the neutral methyl polyethyleneglycol oxycarbonyl-3-amino-1,2-propanediol distearoylester (mPEG-DS) [Garbuzenko O. et al., Langmuir. 21:2560-2568 (2005)]. The PEG moiety preferably has a molecular weight of the PEG head group is from about 750 Da to about 20,000 Da. More preferably, the molecular weight is from about 750 Da to about 12,000 Da, and it is most preferably between about 1,000 Da to about 5,000 Da. One specific PEG-DSPE employed herein is a PEG moiety with a molecular weight of 2000 Da, designated herein ²⁰⁰⁰PEG-DSPE or ² kPEG-DSPE.

Preparation of Liposomes Including Such derivatized lipids has also been described where typically between 1-20 mole percent of such a derivatized lipid is included in the liposome formulation.

Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MVV), and large multivesicular vesicles (LMVV). The liposomes of the first population may be of the same type as those forming the second population or may be of a different type. In accordance with one embodiment, the liposomes of the first population and second population are LMVV. LMVV may be prepared by methods known in the art. For example, LMVV may be prepared by: (a) vortexing a lipid film with an aqueous solution, such as a solution of ammonium sulfate; (b) homogenizing the resulting suspension to form a suspension of small unilamellar vesicles (SUV); and (c) repeatedly freeze-thawing said suspension of SUV in liquid nitrogen followed by water. Preferably, the freeze-thawing is repeated at least five times. The extraliposomal ammonium sulfate is then removed, e.g. by dialysis against normal saline. A therapeutic agent is encapsulated within the liposomes by incubating a suspension of the LMVV liposomes with a solution of the agent. This method is as also described in detail in International Patent Publication No. WO/20000/9089 (the LMVV referred to therein by the abbreviation GMV).

Referring back to the loading methods of the invention, these comprise in general the step of bringing the material to be loaded (an agent or other substances as will be defined below, both, at times, being generally referred to herein by the term “material to be loaded”) into contact with the pre-formed liposomes. Contact may include mixing, suspending, etc. Another step comprises subjecting the pre-formed liposomes to US irradiation. It is noted that the order of steps in the methods of loading is interchangeable. Thus, while in accordance with some embodiments the contacting of the pre-formed liposomes with the material may precede US irradiation, in accordance with some other embodiments of the invention, the pre-formed liposomes are irradiation prior to contact thereof with the material to be loaded. The time span between the US irradiation and contacting of the irradiated liposomes with the material to be loaded therein will depend on the effect of a specific irradiation session on the liposomes' permeability after irradiation is terminated. In other words, the material to be loaded with brought into contact with the liposomes as long as they remain permeable. While typically the liposomes are permeable during US irradiation, the liposomes may be designed such that permeability is retain for a period of time also following irradiation.

In the context of the various aspects of the present invention the term “Ultrasound irradiation” or as used herein at times by the shorter term “irradiation”, denotes the exposure of the liposomes to any ultrasonic wave generated from one or more ultrasonic generating unit (e.g. an ultrasound transducer). The ultrasonic wave may be characterized by one or more of the following parameters: irradiation frequency, irradiation duration, irradiation intensity, number of irradiation sources and sites (locations) per irradiation session (i.e. several irradiations may be applied to different locations within a body), continuous, sequential or pulsed irradiation, focused or non-focused irradiation, uniform or non-uniform (i.e. frequency- and/or amplitude-modulated) irradiation.

In the following description, the ultrasonic wave is characterized by its frequency and duration. However, it is to be understood that the adherence to these two parameters should not be construed in any manner as limiting the invention. For example, the US irradiation may alternatively be defined by its intensity being within the range of between about 0.1 to 10 watt/cm²

According to one embodiment, the ultrasound irradiation is characterized by a frequency of between about 18 kHz to about 1 MHz. A preferred embodiment of the invention provides an US irradiation at a frequency of between about 20 kHz and about 300 kHz, more preferably between about 20 kHz and 100 kHz. This range is recognized at times by the term “low frequency ultrasound irradiation” (LFUS).

The loading methods of the invention may utilize a continuous or pulsed irradiation mode. Further, the loading methods may utilize a series of sequential continuous irradiations. The series of irradiations may be characterized by the same or different irradiation parameters. For example, while the frequency of each irradiation session in the series of irradiations may be the same, the duration of irradiation may vary.

The irradiation, which increases permeability of the liposomes, has been shown herein to positively affect loading of material into the liposomes (which in the absence of irradiation, would not have been loaded into the liposomes).

In the context of the present invention the term “loading” denotes the introduction of the material into the liposome's lipid bilayer, into a single leaflet of the bilayer (e.g. asymetrical loading) or into both leaflets of the bilayer, into the aqueous core of the liposome, or adsorbed to the liposomes' surface (e.g. by ionic interactions of, for example, DNA and siRNA complexes) and combinations of same (i.e. to the leaflet, aqueous core and/or surface). It is well appreciated that irradiating liposomes having substances non-covalently affixed to a liposome's surface may result in the “disordering” of the liposome's membrane and that this “disordering” may lead to the loosening of the substances at the surface, thereby to their release from the liposomes. The material may be fully enclosed within a liposome's compartment (fully embedded in the bilayer and/or encapsulated within the aqueous core), or partially exposed at the outer surface of the liposome (i.e. having part thereof stably anchored within the liposome's outer leaflet or both leaflets).

The compartment into which the material is loaded may depend on the chemical and physical characteristics of the material. For example, loading a hydrophobic (e.g. cholesterol) or amphipathic molecule (e.g. lipid), will mainly be in the lipid membrane.

In accordance with one embodiment of the loading aspect of the invention, the material is referred to by the term “an agent” and the method is conducted for loading the agent for any one of the following applications:
For the preparation of a therapeutic composition, wherein the agent may be drug to be encapsulated within the aqueous core and/or embedded in the membrane;
For the preparation of composition for imaging, where the agent may be a labeled molecule, e.g. fluorophore labeled lipid or radioactive lipid which will be loaded mainly into the liposomes' membrane, or a small molecular weight contrasting agent, which may be encapsulated in the aqueous core;
For modifying the characteristics of the pre-formed liposomes, where the agent may be a modifying lipid, such as a lipopolymer (e.g. PEGylated lipid); a membrane compatible component which may affect fluidity/rigidity of the liposome's membrane and thus permeability, a fatty acid, a charged lipid and lipid like substances which may facilitate in targeting of the liposomes (e.g. for cell transfection). Loading of such substances into pre-formed liposomes may be of advantage when their presence in the liposome prior to or during the loading of a therapeutic agent into the liposomes may reduce the loading efficacy (i.e. their presence may interfere with the drug's loading);
For loading of water soluble molecules or of molecules which are essentially soluble in a physiological medium, in which case the molecules will be introduced into the intra-liposome aqueous phase;
For the loading of macromolecules such as lipids, polymers, polysaccharides;
For incorporation of unstable liposome components (e.g. unstable lipids) to pre-formed liposomes just before their actual use;
For loading of particulate matter, such as nano or micro particles (e.g. carbon nano-tubes), quantum dots, polymer aggregates. In this case, it is a pre-requisite that the particles have a diameter which is smaller than the average diameter of the pre-formed liposome so as to allow effective introduction of the particles into the aqueous compartment of the liposome.

It is noted that hitherto, the composition of liposomes was dictated at the time of liposome preparation. The present invention presents a technology enabling one to ‘plant’ material into liposomes' membrane after the liposome has actually been formed. This permits one to form liposomes of a certain lipid composition, and later, after the liposomes have been formed, to add other materials to the liposome membrane.

This novel concept may be used, for example, to form liposomes of a certain lipid composition, then load a drug into the liposomes by trans-membrane active loading, and only the drug is loaded to add lipids into the liposome membrane. The advantage of such a procedure is in maximizing liposomal drug loading in the case the added lipid interferes with drug loading.

It should be well appreciated by those versed in the art and having understood the loading aspect of the invention that various types of agents may be loaded into the liposomes and thus, the above recitation of some types of agent is for illustration only and should not be regarded in any way as a limiting list of agents to be used in the context of the present invention.

In accordance with another embodiment of the loading aspect of the invention there is provided a method for reducing the amount of a material, referred to as a substance, in a fluid medium. The method in accordance with this embodiment comprises contacting the fluid medium with pre-formed liposomes; and subjecting the pre-formed liposomes to US irradiation, the US irradiation comprises parameters being effective to increase permeability of said liposomes and thereby permit loading of said substance into said liposomes. As indicated above, the order of the steps is not limiting and in principle the said contact of the fluid medium with the pre-formed liposomes may be before or after said irradiation.

The fluid medium may be any medium in which the liposomes' integrity is substantially retained. In accordance with one embodiment, the fluid medium is a biological fluid or any aqueous medium (e.g. solution) requiring purification or cleansing. The term “biological fluid” includes any fluid extracted from a living body (bodily fluid) or from plant material. Biological fluid may comprise any extracellular fluid (ECF), e.g., without being limited thereto, whole blood, blood plasma, blood serum, interstitial fluid, lymph, cerebrospinal fluid, GI tract fluid, synovial fluid, the fluids of the eyes and ears, pleural, pericardial and peritoneal and the glomerular filtrate, etc. as appreciated by those versed in the art.

In accordance with one embodiment, the biological fluid may be extracted from a subject's body, treated ex vivo so as to remove therefrom at least a portion of the substance and then reintroduction of the treated fluid into the same or other subject. Such method may replace conventional kidney dialyses, plasmapheresis procedures, for blood de-toxification as well as for other applications, as may be appreciated by those versed in the art.

In accordance with yet another embodiment of the loading aspect of the invention there is provided a method for reducing the level of a substance in a subject's body (at times referred to herein as the in situ loading method), the method comprises administering to said subject (the blood stream or to an organ or tissue of the subject), an amount of pre-formed liposomes in a manner permitting contact between said liposomes and said substance; and subjecting the pre-formed liposomes to US irradiation, said US irradiation comprises parameters being effective to increase permeability of said liposomes so as to permit loading of said substance into said liposomes, thereby reducing the level of the substance in said subject. Also in this case, the order of the method steps may interchange, such that irradiation of the pre-formed liposomes may take place prior to or shortly after administration of the liposomes to said subject.

It should also be appreciated by those versed in the art that the liposomes after capturing a substance within the subject's body may be removed by conventional methods, such as by dialysis, plasmapheresis, the use of magnetic particles, as well as by natural biochemical processes within the body. The invention should not be limited to a specific mechanism of removal of the loaded liposomes.

The substance within the subject's body may be confined in a specific area or organ or tissue of the subject or free within the subject's body fluids and/or circulatory system. In accordance with one embodiment, the substance is free within the subject's body. The term “free” in the context of this embodiment of the invention is used to denote that the substance is not chemically or physically affixed to a cell, a tissue or organ within the subject's body, i.e. essentially freely moving within the fluid medium in which it is present.

Once within the subject's body irradiation of the pre-formed liposomes requires that the irradiation parameters (as described hereinbefore) are such that essentially no irreversible damage is caused to the subject's body (e.g. tissue or organ) as a result of said irradiation. Damage means an effect that impairs the functionally of the irradiated cell, tissue or organ in an irreversible manner.

The substance in accordance with this in situ loading method of the invention may be any substance which has an undesired biochemical effect within the body or is present at such concentrations which produce (at said concentration) an undesired biochemical effect within the body or its presence within the body is no longer required. This may include, for example and without being limited thereto, a drug (e.g. in case of drug overdose); an imaging agent (after an imaging procedure); a toxic agent (e.g. as a result of poisoning or after being exposed to a toxin or any other chemical compound (e.g. metal containing complexes)); a fatty acid, a lipid, a metabolite, a hormone, a protein, a peptide (e.g. when such a substance is present in the body in unbalanced/high levels); a mineral, etc.

In addition, the invention may also be applicable for the removal of substances within cells and capturing of same by the pre-formed empty liposomes. An example for such an application may relate to the removal of excess of cholesterol or excess of iron such as in thalasemia patients.

It is noted that the body may be irradiated once (a single irradiation treatment) or several times termed herein after “irradiation sessions”. The different irradiation sessions may include a time window between irradiations ranging from several milliseconds to several hours and at times days. Further, when irradiating a specific target within the body, e.g. a specific organ or part thereof, irradiation may be a continuous irradiation or pulsed irradiation (e.g. to avoid overheating of the irradiated target). The target may also be irradiation by the use of a single irradiation source (e.g. a single ultrasonic transducer) or by the use of several sources from different sites being focused on the same target area.

In accordance with the loading aspect of the invention there is also provided a kit comprising a composition of pre-formed liposomes; and instructions for subjecting said composition of pre-liposomes to US irradiation, said instructions identifying irradiation parameters which induce an increase in permeability of the pre-formed liposomes, such that when the irradiated liposomes are brought into contact with an agent, at least a portion of said agent is loaded into said liposomes. When using the kit for in situ loading of a substance, the instructions may also comprise steps required for the preparation of the composition of preformed liposomes for administration to the subject's body, the dose and manner of administration as well as any other instructions required in order to perform the in situ method of the invention.

In accordance with the loading aspect of the invention there is also provided the use of pre-formed liposomes for the preparation of a pharmaceutical composition for removing a substance from a subject's body, said composition being intended for use in combination with exposing said pre-formed liposomes to US irradiation when said composition is within said subject's body.

Referring now to the release aspect of the invention, there is specifically provided a method for the controlled quantum release from liposomes of an agent stably loaded into said liposomes, the method comprises subjecting said liposomes to a series of two or more US irradiation sessions, each US irradiation session comprises parameters being effective to increase permeability of said liposomes thereby permitting release of a predetermined amount of said agent from said liposomes at same site or at different body sites.

The term “controlled quantum release” as used herein denotes the step-wise release of an amount of the agent from the liposomes to the liposomes' surrounding, the amount being controlled by the use of a specific membrane composition and/or the selected irradiation parameters. The stepwise release also denotes that the amount of agent released in each irradiation session is a fraction of the initial total amount of the agent within the liposome. For example, the controlled quantum release may be designed such that in a series of 10 irradiations, about 10% of the total amount of the agent is released in each irradiation session. Alternatively, according to the therapeutic regime the release may be tailored so that in each irradiation session a different amount of agent is released, either according to pre-defined plan, or according to clinical parameters tested in the individual. In order to facilitate such a release profile, the irradiation sessions need not to be defined by the same parameters. For example, in order to control the quantum release of the agent from the liposomes in accordance with a predefined profile the first irradiation session may the shortest (e.g. milliseconds) and each following irradiation session may be of a slightly longer duration. The frequencies may also vary between irradiation sessions as well as other irradiation parameters.

The release methods of the invention may be applicable for, inter alia, the release of water soluble molecules or of molecules which are essentially soluble in a physiological medium, in which case the molecules are encapsulated in the intra-liposome aqueous phase; for the release of macromolecules such as lipids, polymers, polysaccharides etc. which may be incorporated in the lipid membrane and/or intraliposome aqueous phase; for the release of particulate matter, such as nano or micro particles (e.g. carbon nano-tubes), quantum dots, polymer aggregates., etc. In the latter case, it is a pre-requisite that the particles have a diameter which is smaller than the average diameter of the pre-formed liposome so as to permit encapsulation of the particles in the aqueous compartment of the liposome.

It is noted that in order to facilitate the controlled quantum release of the agent from the liposome, a pre-requisite is that the agent is stably loaded within the liposomes. Stable loading denote that no more than 10% of the agent is released from the liposome during storage at 4° C. for a period of at least one month. The time period between irradiations in the series of two or more irradiation sessions may vary from several miliseconds, several hours to several days. It is known that liposomes may at times reside in blood circulation with half life time of more than 70 hours and in target tissues half life time as much as 200 hours following administration. Thus, the method in accordance with the release aspect of the invention may include a schedule of several administrations of liposomes each followed by a series of two or more irradiation sessions.

Further in accordance with the release aspect of the invention there is provided a kit comprising a composition of liposomes encapsulating an agent; and instructions for applying a series of two or more US irradiation sessions on a subject's body following administration of said composition of liposomes to said subject, said instructions comprising an index identifying irradiation parameters for each irradiation session and the amount of agent released from said liposomes during an identified irradiation session.

Alternatively, there is provided a kit comprising a composition of liposomes encapsulating an agent; and instructions for applying a series of two or more US irradiation sessions to a subject's body following administration of said composition of liposomes to said subject, said instructions comprise an index of treatment protocols corresponding to patient and disease-related parameters, the treatment protocols defining irradiation parameters.

The index may be provided in various forms. In accordance with one embodiment, the index may be provided in the form of a calibration curve or a table plotting the percent/amount of release of the agent from the liposomes as a function of irradiation parameters.

Throughout the description and claims of this specification, the singular forms “a” “an” and “the” include plural references unless the context clearly dictates otherwise. Thus, for example, a reference to “a liposome” is a reference to one or more liposomes and “an agent” refers to one or more agents. Throughout the description and claims of this specification, the plural forms of words include singular references as well, unless the context clearly dictates otherwise.

Yet, throughout the description and claims of this specification, the words “comprise” and “contain” and variations of the words, for example “comprising” and “comprises”, mean “including but not limited to”, and are not intended to (and do not) exclude other moieties, additives, components, integers or steps.

The invention will now be described by way of non-limiting examples.

### DESCRIPTION OF SOME NON-LIMITING EXAMPLES

### Example 1

### Loading into Liposomes Using Ultrasound (US)

Low frequency US Effect on Membrane Integrity and Pyranine Uptake

In the following experiment the uptake of the highly hydrophilic, membrane-impermeable fluorescent compound pyranine (Molecular Probes, Eugene, Oreg.) into SSL was followed. For this, pyranine was added to the external medium of the SSL as described by Avnir et al. [Avnir, Y.; Barenholz, Y., Anal. Biochem. 2005, 347 (1), 34-41]. Then SSL dispersions were exposed to LFUS (3.3 W/cm²) and the anion exchange resin Dowex 1×8 was added to remove non-liposomal pyranine. The determination of intraliposomal pyranine was based on the fluorescence emission intensity at 507 nm, the pH-independent isosbestic point (excitation at 415 nm) [Bing, S. G.; et al. Am. J. Physiol. Cell Physiol. 1998, 275 (4), C1158-C1166]. Fluorescence measurements were conducted in the presence of the membrane-impermeable fluorescence quencher, DPX (p-xylene-bis-pyridinium bromide, Molecular Probes) in order to decay fluorescence of any residual non-liposomal pyranine [Clerc, S.; Barenholz, Y., Biochim. Biophys. Acta, Biomembranes 1995, 1240 (2), 257-265] as will be discussed below, liposome permeability was transiently increased during exposure to LFUS, thus inducing loading of pyranine into the interliposomal aqueous compartment.

Cryogenic Transmission Electron Microscopy (cryoTEM)

CryoTEM work was performed at the Hannah and George Krumholz Laboratory for Advanced Microscopy (Technion, Haifa, Israel). For each experiment, lipid dispersions at concentrations of 50 and 5 mM in 5% (w/v) dextrose in a total volume of 400 μL were used. Specimens were prepared in a controlled-environment vitrification system at 25° C. and 100% relative humidity and examined in a Philips CM120 cryo-electron microscope operated at 120 kV. Specimens were equilibrated in the microscope below −178° C., then examined in the low-dose imaging mode to minimize electron beam radiation damage, and recorded at a nominal under-focus of 4-7 nm to enhance phase contrast [Simberg, D.; et al. J. Biol. Chem. 2001, 276 (50), 47453-47459]. An Oxford CT-3500 cooling holder was used. Images were recorded digitally by a Gatan MultiScan 791 CCD camera using the Digital Micrograph 3.1 software package.

### Results

### LFUS Transiently Ruptures the Liposome Membrane

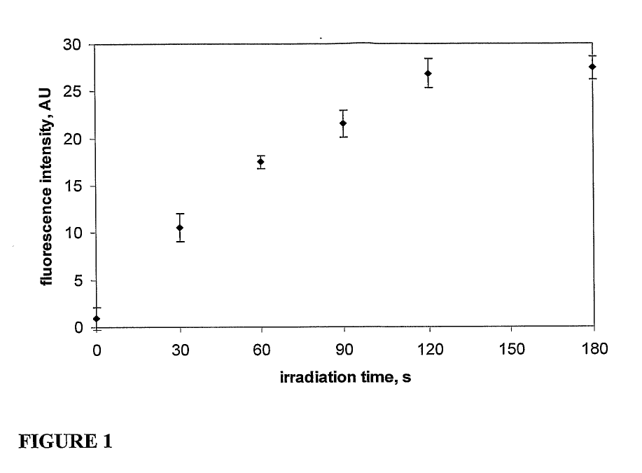

Based on the results obtained and without being bound to a specific mechanism, it is thus proposed that LFUS induces a transient disruption of the liposome lipid bilayer, releasing loaded drug. If this is the case, then LFUS may also cause leakage of extraliposomal medium solutes into the intraliposomal aqueous compartment. This was tested by adding a water-soluble highly negatively-charged, membrane-impermeable fluorophore, pyranine, to the extraliposomal aqueous medium prior to irradiation. Then the liposomal dispersion was irradiated, and the level of pyranine in the intraliposomal aqueous compartment was quantified. FIG. 1 shows that pyranine is taken up into the liposomal aqueous compartment, having an uptake level proportional to the exposure time of SSL to LFUS.

These findings support the hypothesis of transient liposome membrane rupture and/or formation of pore-like membrane defects as the mechanism of LFUS-induced rapid drug release, followed by rearrangement/resealing of the lipid bilayer. The first-order release kinetics data (see above discussion) also support such a mechanism.

### Example 2

### Introducing Lipids and Other Substances into Liposomes by LFUS

The following example presents the introduction, using LFUS, of lipids into the liposome membrane after the liposomes have been formed.

### Material and Methods

HSPC (hydrogenated soybean phosphatidylcholine, Lipoid, Ludwigshafen, Germany) 48 mM, cholesterol (Sigma, St. Louis, Mo.) 36 nM, and OG-PE—(Oregon Green—1,2-dihexadecaneyl-sn-glycero-3-phosphoethanolamine [DMPE], Molecular Probes) 0.336 μmol per 2 mL liposome dispersion, were dissolved in absolute ethanol at 70° C., and then rapidly injected into a calcium acetate (Sigma) aqueous buffer (200 mM, pH 6.5) at an ethanol to buffer ratio of 1:10 (by vol), to form multilamellar vesicles. These were downsized, by stepwise extrusion, through polycarbonate membranes (Osmonics, Trevose, Pa.) using a Lipex extruder (Northern Lipids, Vancouver, Canada), to a diameter of 400 nm. 300 μL of the liposomal dispersion was placed in HPLC glass sample vials (0.5 mL) and held in a water bath at RT.

Twenty mol % of the cationic lipid 1,2-dioleoyl-3-trimethylammonium-propane chloride (DOTAP) or of the anionic lipid 1,2-dimyristoyl-sn-glycero-[-phospho-rac-(1-glycerol)]sodium salt (DMPG), both from Avanti Polar Lipids (Alabaster, Ala.), diluted in ethanol, was then added to the liposomal dispersion.

The sample was then irradiated by LFUS (20 kHz, VC400 Sonics and Materials, Newtown, Conn.) at 4.2 W/cm² for 60 seconds using a 13-mm diameter probe held at a distance of ˜5 mm from the sample vial.

### Analysis

Aliquots of 10 μL of each sample were diluted in 1 mL of 20 mM HEPES (pH 7.5) and analyzed for size and zeta-potential (3 times, 30 sec each) at 25° C. using a Zetasizer Nano-Z, Malvern Instruments, UK.

### Results

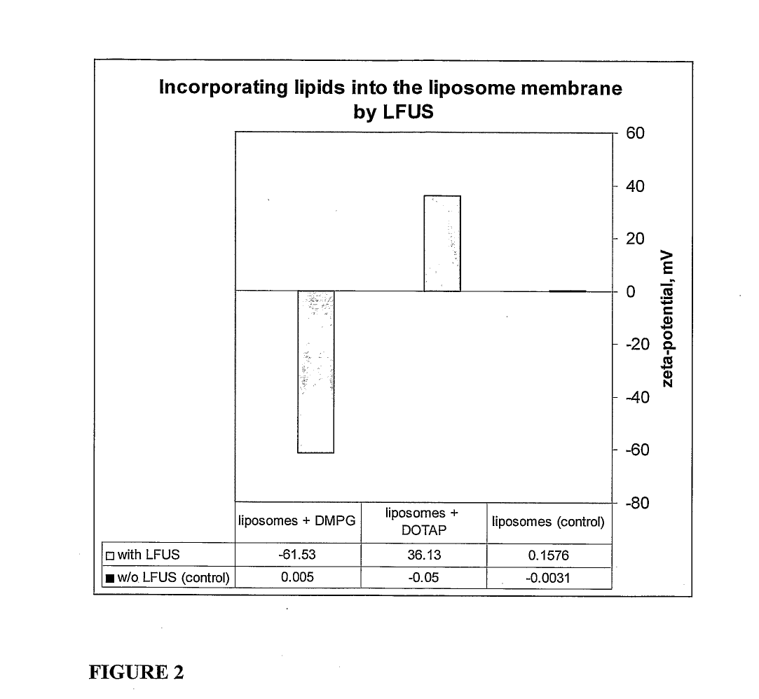

FIG. 2 shows that the zeta-potential of liposomes incubated with the cationic lipid DOTAP (−0.05 mV), or with the anionic lipid DMPG (0.005 mV) were similar to that of the control (−0.0031 mV).

On the other hand, a significant zeta-potential change was measured in liposomes incubated with cationic or anionic lipids and exposed to LFUS. For liposomes incubated with the cationic lipid DOTAP, the zeta-potential raised to a value of 36.13 mV after exposure to LFUS, and liposomes incubated with DMPG and exposed to LFUS showed a zeta-potential value of −61.53 mV. These data indicate that LFUS is capable of incorporating lipids into the liposomal membrane, even after the liposomes were formed.

Furthermore, it must be noted that the mean diameter of LFUS treated and non-treated liposomes was found to be similar (±3%) and that the zeta-potential of liposomes exposed to LFUS but not to charged lipids remained at baseline (control).

It may thus be concluded that LFUS is an effective tool to facilitate incorporation of lipids into the liposome membrane even a long time after liposome formation.

### Example 3

### Controlled Release of Drug from Liposomes Using Ultrasound (US)

### Materials and Methods

### Liposome Preparation

Hydrogenated soybean phosphatidylcholine (HSPC), Mw 750, (Lipoid, Ludwigshafen, Germany) 51 mol %, polyethylene glycol distearoyl phosphoethanolamine (m²⁰⁰⁰PEG-DSPE), Mw 2774, (Genzyme, Liestal, Switzerland) 5 mol %, and cholesterol (Sigma, St. Louis, Mo.) 44 mol % were dissolved in absolute ethanol (Gadot, Haifa, Israel) at 62-65° C. (above the lipid phase transition temperature Tₘ of HSPC, 53° C.). This was added to an aqueous solution, at 62-65° C., of either calcium acetate, ammonium sulfate, or cisplatin, to form multilamellar vesicles (MLV) as described [Pons, M.; et al. Int. J. Pharm. 1993, 95(1), 51-56]. The MLV were downsized to form small unilamellar vesicles (SUV) by stepwise extrusion through polycarbonate membranes (Osmonics, Trevose, Pa.) using a Lipex extruder (Northern Lipids, Vancouver, Canada) starting at a pore diameter of 400 nm and ending at 50 nm.

Thus, all liposomal formulations used in this study were sterically stabilized (SSL), identical in lipid composition (HSPC/cholesterol/mPEG-DSPE) and size distribution (˜100 nm), but differed in the encapsulated drug and drug loading method.

### Drug Loading

### Methylprednisolone Hemisuccinate Sodium Salt (MPS)

Methylprednisolone hemisuccinate sodium salt (MPS), Mw 496.53, (Pharmacia, Puurs, Belgium) a highly potent anti-inflammatory steroid, being a weak acid (pKa 4.65), was remote loaded into liposomes using a high intraliposome/low extraliposome (medium) transmembrane calcium acetate gradient, previously developed [Clerc, S.; et al. Biochim. Biophys. Acta, Biomembranes 1995, 1240 (2), 257-265] and recently adapted for remote loading of MPS [see International patent application publication WO2006/027787].

For preparation of SSL-MPS (MPS loaded into sterically stabilized liposomes), lipids were hydrated in a calcium acetate (200 mM), dextrose (5%, w/v) aqueous solution (pH 6.5) to form MLV, and then downsized to form ˜100-nm SUV by extrusion (see 2.1). The transmembrane calcium acetate gradient was created by replacing non-liposomal calcium acetate with 5% dextrose (pH 4.0), by dialysis. Then MPS was loaded into the liposomes by incubating the liposome dispersion for 1 h at 62-65° C. in a solution of 8 mg/mL MPS in 5% dextrose. Non-loaded MPS was removed by dialysis against 5% dextrose and/or by the anion exchanger Dowex 1×8 (Sigma).

The final MPS-SSL had a drug-to-phospholipid mole ratio of ˜0.33.

### Doxorubicin

The anti-cancer liposomal drug Doxil in which the chemotherapeutic agent doxorubicin, an amphipathic weak base, is remote loaded into SSL utilizing a high intraliposome/low extraliposome ammonium sulfate gradient [Haran, G.; et al. Biochim. Biophys. Acta, Biomembranes 1993, 1151 (2), 201-215] was used. Mean liposome diameter was ˜100 nm and drug-to-lipid mole ratio was ˜0.3.

Doxil, a gift of ALZA (Mountain View, Calif.), was supplied as an isotonic suspension containing 2 mg doxorubicin per mL of 10 mM histidine buffer, pH 6.5, with 10% w/v sucrose.

### Cisplatin

SSL passively loaded with the anti-cancer chemotherapeutic agent cisplatin (“Stealth” cisplatin) was prepared as described by Peleg-Shulman [Peleg-Shulman, T.; et al. Biochim. Biophys. Acta, Biomembranes 2001, 1510 (1-2), 278-291]. Mean liposome diameter was ˜110 nm and drug-to-lipid mole ratio was ˜0.032.

Stealth cisplatin, a gift of ALZA, was supplied as an isotonic suspension of 1 mg/mL cisplatin in 10% w/v sucrose, 1 mM sodium chloride, and 10 mM histidine buffer, pH 6.5.

Table 1 summarizes the three drug loading parameters:

TABLE 1
Drug-liposome characteristics

| Liposomal | | | |
|---|---|---|---|
| drug | MPS | Doxorubicin | Cisplatin |
| Chemical | Methylprednisolone | Doxorubicin- | Cisplatin |
| name | hemisuccinate | HCl | |
| | sodium salt | | |
| Drug | Amphiphilic weak | Amphiphilic | Non-amphiphilic, |
| character- | acid | weak | Low water |
| ization | | base | solubility |
| Loading | Active - calcium | Active - | Passive - |
| method | acetate gradient | Ammonium | Liposome |
| | | sulfate | formation at 65° C. |
| | | gradient | with drug |
| | | | dissolved in the |
| | | | hydrating liquid |
| Drug/ | 0.33 | 0.30 | 0.032 |
| Lipid | | | |
| (mole | | | |
| ratio | | | |

| | |
|---|---|
| Lipid | Hydrogenated soybean phophatidylcholine (HSPC) 51 mol %, |
| compo- | polyethylene glycol distearoyl phosphoethanolamine |
| sition | (mPEG2000-DSPE) 5 mol %, and cholesterol 44 mol % |
| Diameter | ~100 nm |

### Ultrasound Apparatus

In vitro

A 20-kHz low-frequency ultrasonic processor, LFUS, (VC400, Sonics & Materials, Newtown, Conn.) was used. The ultrasonic probe (13-mm diameter) was immersed in a glass scintillation vial containing 3 mL of liposome dispersion. Irradiation was conducted at a full duty cycle at varying intensities (from 0 to 7 W/cm²) and durations (0 to 180 s). The sample vial was kept in a temperature-controlled water bath and its temperature was monitored (37° C.) throughout the experiment to prevent heat-induced liposomal drug release [Maruyama, K. et al. Biochim. Biophys. Acta 1993, 1149 (2), 209-16; Sharma, D. et al. Melanoma Res. 1998, 8 (3), 240-244; and Unezaki, S. et al. Pharm. Res. 1994, 11 (8), 1180-5].

### In Vivo

Tumor induction. One million J6456 lymphoma tumor cells suspended in 200 μL of serum-free phosphate buffered saline (PBS), pH 7.4, were injected intraperitoneally (i.p.) into thirteen 8-week-old BALB/c female mice (Harlan Labs, Jerusalem, Israel) [Gabizon, A.; et al. Adv. Drug Delivery Rev. 1997, 24 (2-3), 337-344]. One week after cell inoculation abdominal tumors were observed. Animals were divided into three test groups: (i) control (placebo), (ii) liposomal cisplatin without LFUS, and (iii) liposomal cisplatin plus LFUS.

Drug treatment. Liposomal cisplatin groups (ii and iii) were administered i.p., directly into the tumor to a depth of ˜2 cm, 2 mL of the liposome dispersion (15 mg drug per kg body weight) in PBS. The control group was treated with 2 mL PBS. Then, all three groups were anesthetized in an ether bath and sacrificed two hours later. The drug was extracted from the tumor and quantified (see below).

Ultrasound treatment. After drug treatment, animals in the group treated with liposomal cisplatin plus LFUS were anesthetized, and the abdominal fur over the tumor was removed. A rubber cylinder, open at both ends, was sealed to the abdomen over the tumor using a silicone paste (Bayer) and filled with water. The LFUS probe was inserted into the water-filled cylinder ˜5 mm above the skin. LFUS irradiation was conducted at an intensity of 5.9 W/cm² for 120 s.

Determination of liposome-encapsulated and released cisplatin. Two hours after treatment, animals were sacrificed and 2 mL PBS was injected into the tumor, and the abdominal area was massagued to free tumor cells and extracellular fluids. Tumor fluids were aspirated using a syringe and centrifuged to separate cells from extracellular fluids. The extracellular fluids were chromatographed by gel permeation chromatography (GPC) to separate released cisplatin from non-released, liposome encapsulated, cisplatin (SSL-cisplatin). The GPC fractions and the tumor cells were quantified for cisplatin by atomic absorption specroscopy (AAS) (see section 2.4.5.2).

### Analytical Procedures

### Evaluation of Liposome Lipid Integrity by Thin Layer Chromatography

Thin layer gel chromatography (TLC) was used to determine if any chemical changes were induced in the liposome lipids by exposure to ultrasonic irradiation. Lipids of liposomal dispersions before and after ultrasonic irradiation were extracted by the Bligh and Dyer procedure [Bligh, E. G.; and Dyer, W., Can. J. Biochem. Physiol. 1959, 37, 911-917] and analyzed by TLC (silica gel 60, Merck, Darmstadt, Germany), which was developed using a solvent system of chloroform/methanol/water (65:25:4 by vol). Spot detection was performed by spraying plates with 1.6 M copper sulfate (Sigma) in 6.8% phosphoric acid, v/v, (BioLab, Jerusalem, Israel) and drying the plates with warm air [Brailoiu, E. et al. Biomed. Chromatograph. 1994, 8 (4), 193-5; Rodriguez, S. et al. Lipids 2000, 35 (9), 1033-1036; and Barenholz, Y. and Amselem, S., Quality control assays in the development and clinical use of liposome-based formulations in Liposome Technology. 2nd ed.; CRC: Boca Raton, Fla., 1993].

### Liposome Size Distribution Analysis

Liposome size distribution before and after LFUS irradiation was measured by dynamic light scattering (DLS) using an ALV-NIBS/HPPS particle sizer equipped with an ALV-5000/EPP multiple digital correlator, at a scattering angle of 173° (ALV, Langen, Germany). These measurements were confirmed by DLS at three other angles (30°, 90°, 150°) using the ALV/CGS-3 Compact Goniometer System (ALV). For the latter, intensity of the DLS signal was also measured.

Determination of Liposomal Cisplatin Level by GPC Combined with AAS

Ultrasonically irradiated and non-irradiated liposomal cisplatin dispersions were chromatographed using GPC on wetted Sephadex G-50 fine (Pharmacia, Uppsala, Sweden) packed in 5-mL polypropylene columns (diameter 1 cm) (Pierce, Rockford, Ill.), and excess column water was removed by centrifugation at ˜580 g. Then, aliquots of 150 μL of liposomal dispersions were applied to the column and centrifuged at ˜580 g. SSL were collected at the void volume [Druckmann, S. et al. Biochim. Biophys. Acta 1989, 980 (3), 381-4; Gabizon, A. et al. Clin. Pharmacokinet. 2003, 42 (5), 419-436] and SSL phospholipids were quantified using the modified Bartlett method [Gabizon, A. et al. Clin. Pharmacokinet. 2003, 42 (5), 419-436 and Bartlett, G. R.,. J Biol. Chem. 1959, 234 (3), 466-468]. Cisplatin level was determined by AAS (see 2.4.5.2 below) and drug-to-lipid mole ratio was calculated.

### Cisplatin Biological Activity

Cytotoxicities of SSL cisplatin and of cisplatin released from SSL by exposure to LFUS and of free cisplatin were tested on cisplatin-sensitive C26 murine colon adenocarcinoma cells. Cell medium consisted of RPMI 1640 with L-glutamine 90%, fetal calf serum (virus-screened) 9% and penicillin-streptomycin solution 1% (all from Biological Industries, Beit Haemek, Israel). Aliquots of 800 cells per well were plated in 96-well plates (Nunc, Roskilde, Denmark) and incubated under 5% CO₂ at 37° C. for 24 h. Equal amounts (15 μL) of non-irradiated and LFUS irradiated SSL cisplatin dispersions, as well as free non-irradiated cisplatin (0 to 45 μL of 1 μg/mL cisplatin (Sigma) in saline), were added to separate wells and incubated for 24 h. Then, surviving cells were fixed by incubating for 15 min with glutaraldehyde (Sigma) 2.5%, v/v, in water. Non-fixed cells were washed away with water and then with 0.1 M boric acid buffer (Sigma), pH 8.7, and stained with 1% methylene blue (Sigma) by incubation for 1 h at 37° C. After treatment, excess stain was washed from the wells. Cell survival was quantified by measuring absorbance at 620 nm after addition of 0.1 M HCl [Oliver, M. H. et al. J. Cell Sci. 1989, 92 (3), 513-518].

### Drug and Gradient Quantification

Immediately after LFUS irradiation, the released drug was removed, and the drug remaining in the liposomes was quantified.

Released drugs were adsorbed using ion exchange resins: MPS on Dowex 1×8 anion exchanger (Sigma) and doxorubicin on Dowex 50W cation exchanger (Sigma) [Storm, G.; et al. Biochimn. Biophys. Acta, Biomembranes 1985, 818 (3), 343-351]. LFUS-released cisplatin was removed by gel exclusion chromatography using Sepharose 6B (Sigma) [Diederichs, J. E., Electrophoresis 1996, 17 (3), 607-611; Mora, R.; et al. J. Lipid Res. 1990, 31 (10), 1793-1807]. Levels of drugs remaining in the SSL were quantified by HPLC for MPS, fluorescence for doxorubicin, and AAS for cisplatin (see below).

### MPS

MPS concentration and chemical integrity were determined using HPLC (Hewlett Packard Liquid Chromatograph 1090). ChemStation software (Hewlett Packard) controlled all modules and was used for analysis of the chromatography data. The analytical column used was a C18 5-micron Econosphere, length 150 mm, inner diameter 4.6 mm (Alltech, Carnforth, UK). Sample injection volume was 20 μL. Eluent was monitored at a wavelength of 245 nm with a 10 nm bandwidth. The mobile phase, acetate buffer, pH 5.8, and acetonitrile (67:33, v/v) was delivered at a flow rate of 1 mL/min [Smith, M. D., J. Chromatogr. 1979, 164 (2), 129-137; Smith, M. D.; et al. J. Chromatogr. 1979, 168 (1), 163-9]. MPS mean elution time was at ˜2.9 min, and the sample run time was ˜5 min.

### Cisplatin

Integrity analysis of ultrasonically released cisplatin was performed by ¹⁹⁵Pt NMR spectroscopy. Experiments were performed on an INOVA 500-MHz spectrometer (Varian, Palo Alto, Calif.) using standard pulse sequences. The Pt chemical shifts were assigned relative to the external reference signal of K₂PtCl₄, set at −1,624 ppm. A line broadening of 300 Hz was normally applied, and data were processed using the VNMR software (Varian) [Peleg-Shulman, T.; et al. Biochim. Biophys. Acta, Biomembranes 2001, 1510 (1-2), 278-291].

Cisplatin was quantified by AAS of Pt, at 2700° C. (λ=265.9 nm), using a Zeeman atomic absorption spectrometer SpectAA300 (Varian) in reference to a standard Pt solution (BDH Chemicals, Poole, UK).

### Doxorubicin

Doxorubicin was quantified by determining the fluorescence emission intensity at 590 nm (excitation 480 nm), in reference to a doxorubicin standard curve, after disintegrating the liposomes in acidic isopropanol (0.075 N HCl), [Gabizon, A.; et al. Cancer Res. 1994, 54 (4), 987-992] using an LS50B luminescence spectrometer (Perkin Elmer, Wellesley, Mass.), equipped with WinLab PE-FL software (Perkin Elmer).

### Acetate Gradient

Levels of LFUS-released acetate, as well as intraliposome acetate, were determined enzymatically using the Megazyme (Wicklow, Ireland) acetic acid assay kit. Determination of intraliposome acetate required liposome dissolution by ethanol, and therefore the acetic acid standard curve was made with the same amount of ethanol as in the analyte [WO2006/027787].

### Results

### Effect of Ultrasound Amplitude on Release of Drugs

Initial tests verified the dependence of liposomal drug release on the ultrasonic amplitude. Drug-loaded liposome dispersions were irradiated by LFUS for 60 s [this irradiation duration was selected with the intention to be at the part of the curve in which drug release did not reach a plateau]. Amplitude of irradiation was increased from sample to sample, in the range of 0 W/cm² (no irradiation, i.e. control) to 7 W/cm².

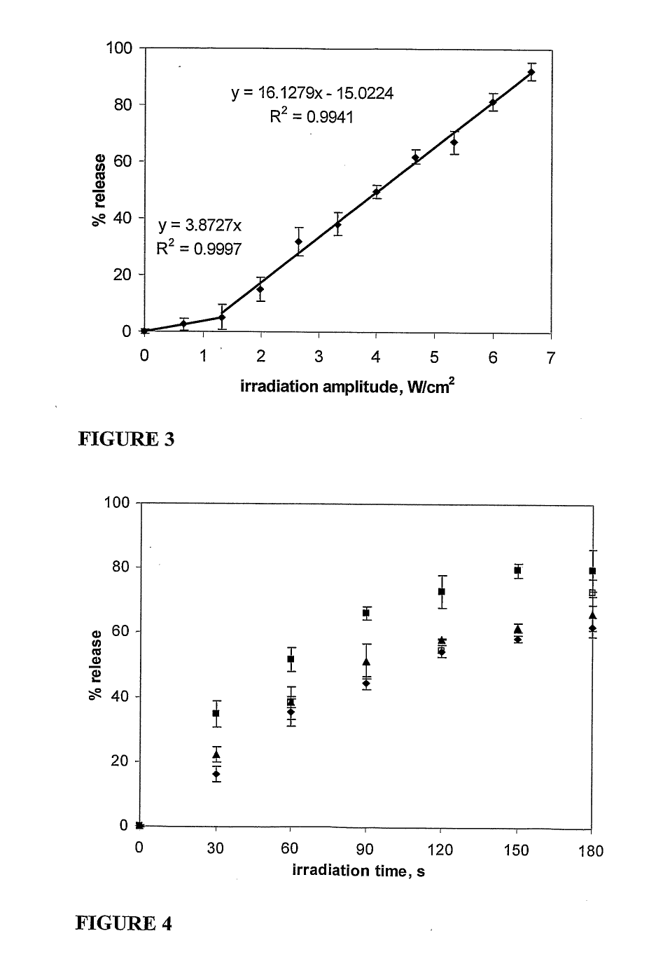

FIG. 3 shows that the dependence of liposomal MPS release on the ultrasonic amplitude is biphasic. Both phases are linear, but differ in their slopes, a low slope (˜3.9 [% release/(W/cm²)]) up to the amplitude of ˜1.3 W/cm², and a higher slope (˜16.1) above this amplitude. The increase in drug release above ˜1.3 W/cm² is explained by the initiation of a transient cavitation (i.e., the formation, growth, and implosive collapse of bubbles in a liquid) above this energetic threshold [Mitragotri, S.; Kost, J., Adv. Drug Delivery Rev. 2004, 56 (5), 589-601].

Thus, it is suggested that cavitation occurs near the liposome membrane, in the extraliposomal medium and/or by small cavitation nuclei in the intraliposomal aqueous compartment.

Non-irradiated SSL containing each of the three drugs (doxorubicin, cisplatin and MPS) released <3% of the loaded drug over the experimental period, when kept at 37° C. It is further noted, that non-irradiated SSL exhibited less than 10% drug leakage over a period of 6 months (data not shown).

### Effect of Irradiation Time on Level of Release

SSL containing the drugs doxorubicin, cisplatin, or MPS, or SSL having a high intraliposome/low extraliposome acetate gradient (the driving force for remote loading of MPS), were irradiated by LFUS at constant amplitude (3.3 W/cm²) for different periods of time, from 0 to 180 s.

For SSL loaded with MPS, 80% of the drug was released within the first 150 s of irradiation, after which drug release plateaus. The other formulations, doxorubicin, cisplatin, and acetate, had similar curve characteristics, but slightly lower release levels (FIG. 4).

These data show that substantial release of liposomal drugs can be obtained by short-term exposure to LFUS. This effect was thus defined as “dumping”, meaning release of the majority of the encapsulated drug within a short period of time, creating a high concentration of the drug in the vicinity of the irradiated SSL.

Analyzing drug release data revealed that ultrasonically-triggered liposomal drug release (up to ˜150 s), at a fixed ultrasound amplitude, follows first-order kinetics:

−dA/dt=k*A

or in its integrated form: log(A₀/A)=k*t, where dA/dt is the change in concentration with time, k is the first order rate constant, A₀ is the initial amount of drug loaded in the liposomes, and A is the remaining amount of drug in the liposomes after an irradiation time t, indicating, that for a given liposomal drug, release is dependent on irradiation time.

The following first-order rate constants (k) were determined for LFUS-induced release: 0.0053 s⁻¹ for MPS, 0.0029 for cisplatin, 0.0033 for doxorubicin, and 0.0031 for acetate (R²=0.994, 0.995, 0.992, and 0.992, respectively).

### Affect of LFUS Irradiation Profiles on Drug Release

### Pulsed Release

Liposomal dispersions containing MPS were irradiated at an amplitude of 3.3 W/cm² for different periods of time, comparing drug release of samples that were irradiated continuously with those irradiated by pulsed mode for the same accumulated irradiation time.

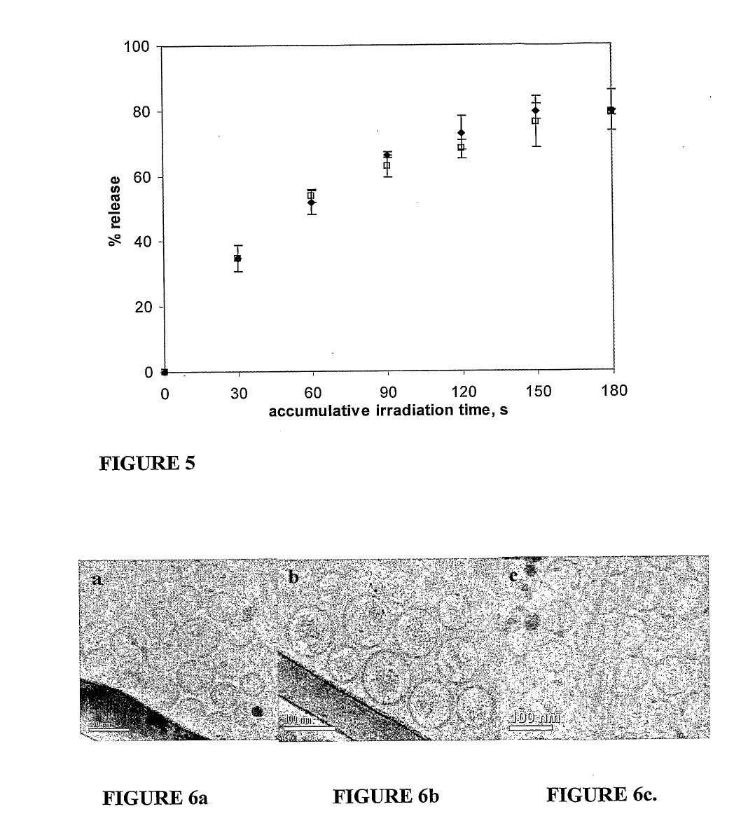

FIG. 5 shows that the drug release profile of continuous and pulsed LFUS modes are almost identical with respect to the actual time of exposure to LFUS, indicating that drug release depends only on the actual irradiation time and that the effect of irradiation on liposomal drug release is cumulative. Therefore, irradiation can be conducted either at continuous or at pulsed mode to obtain the same drug release. These results are important for clinical applications, where several repeated short exposures are usually preferred to one long exposure in order to prevent heating-related damage to tissue.

### Drug Release Occurs Only During the Actual LFUS Exposure Time

It is well established that LFUS is capable of increasing permeability of biological membranes, and permeability increase is retained for a long time after irradiation has ended [Kost, J.; Langer, R.,. J. Acoust. Soc. Amer. 1989, 86 (2), 855; Duvshani-Eshet, M.; et al. Gene Ther. 2006, 13 (2), 163-172; Rapoport, N.; et al. Arch. Biochem. Biophys. 1997, 344 (1), 114-124]. It was now shown that LFUS is capable of increasing the permeability of the liposome membrane, enabling drug release. It was tested whether the permeability increase was prolonged or confined only to the irradiation period.

For this, liposomal dispersions were irradiated at 3.3 W/cm² for periods of 30 to 180 s, and drug release was determined immediately after irradiation and 72 h later.

Levels of released drug were the same at both times (data points coincide, not shown). This indicates that increased permeability of the liposome membrane is transient and occurs only during exposure to LFUS, and that after irradiation is terminated the liposome membrane becomes impermeable again and drug release stops.

Combining these results with the data shown for pulsed release suggests that LFUS can be used for controlling the level of drug release over prolonged periods of time, which is very important for successful drug delivery.

### CryoTEM Analysis of Liposome Structure

The structure of liposomes before and after exposure to LFUS was examined by cryo-transmission electron microscopy (cryoTEM).

FIG. 6a presents liposomes before loading with MPS and before exposure to LFUS. The liposome membrane is clearly noticed as the slightly darker perimeter of the liposomes surrounding the inner aqueous compartment. FIG. 6b presents SSL remote loaded with MPS by means of a calcium acetate transmembrane gradient. The loaded drug, most likely as calcium MPS precipitate, appears as the darker area within the SSL aqueous compartment. FIG. 6c presents liposomal MPS irradiated for 120 s at 3.3 W/cm². No change in the appearance of the liposome membrane or size was noticed after irradiation. In all cases, the liposome diameter indicated by cryoTEM correlates well with the DLS measurements (presented in 3.3.5).

However, LFUS seems to have a great effect on the appearance of the intraliposomal MPS precipitate. While non-irradiated SSL show massive precipitate in the intraliposomal aqueous phase (FIG. 6b), irradiated liposomes (FIG. 6c) seem to be either empty or to have much less precipitate, which accords with the release of MPS by exposure to LFUS, as shown in FIG. 4.

Without being bound to theory, these findings suggest that LFUS induces transient porous defects in the liposome membrane, enabling drug release, which occurs only during the exposure to LFUS, after which, membrane integrity is restored.

### LFUS Induces Disruption of a Fraction of the Liposomes

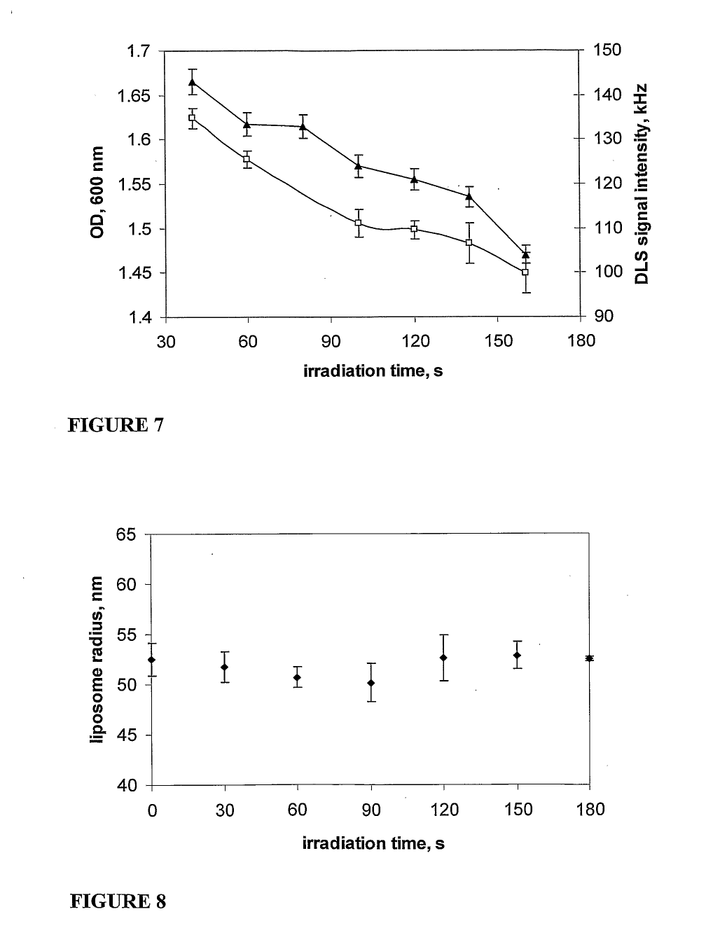

It was now found that the turbidity of liposomal dispersions decreased as LFUS irradiation time increased. This effect occurred in all three liposomal drug formulations, doxorubicin, cisplatin, and MPS, and also in drug-free liposomes (exemplified in FIG. 7 for MPS).

Without being bound to theory, a possible explanation of such a change in turbidity is a decrease in liposome size [Woodbury, D. J.; et al. J. Liposome Res. 2006, 16 (1), 57-80; Pereira-Lachataignerais, J.; et al. Chem. Phys. Lipids 2006, 140 (1-2), 88-97]. This assumption was tested by measuring the diameter of LFUS-irradiated liposomes using dynamic light scattering (DLS) at four different angles (30°, 90°, 150°, and 173°; the use of wide and narrow angles was conducted to more sensitively test for the presence of smaller or larger liposomes, respectively [Hiemenz, P. C., Principles of Colloid and Surface Chemistry. 3rd ed.; Marcel Dekker: New York, 1997; Beme, B. J.; Pecora, R., Dynamic Light Scattering. John Wiley and Sons: New York, 2000]). The results indicated that the liposome diameter remained unaffected, independent of irradiation time (see FIG. 8 for DLS data at 90°). Therefore, it is proposed herein that the decrease in turbidity is not due to a decrease in SSL diameter or drug release, but rather due to a decrease in the number of liposomes in the dispersion, by disassembly of some of the liposomes.

This assumption was tested in two different ways: (i) by recording the DLS signal intensity of liposomal dispersions irradiated for different times. (ii) by direct evaluation of the liposome phospholipid concentration relative to the total phospholipid concentration (liposomal plus non-liposomal) in dispersions exposed to LFUS for different times.

For liposome preparations of identical size distribution, the DLS signal intensity is proportional to the concentration of liposomes present in each dispersion (number of liposomes per unit volume) [Hiemenz, P. C., Principles of Colloid and Surface Chemistry. 3rd ed.; Marcel Dekker: New York, 1997; Benie, B. J.; Pecora, R., Dynamic Light Scattering. John Wiley and Sons: New York, 2000] The decrease in the DLS signal intensity with irradiation time (shown in FIG. 7) suggests a decrease in the concentration of liposomes present in the dispersion.

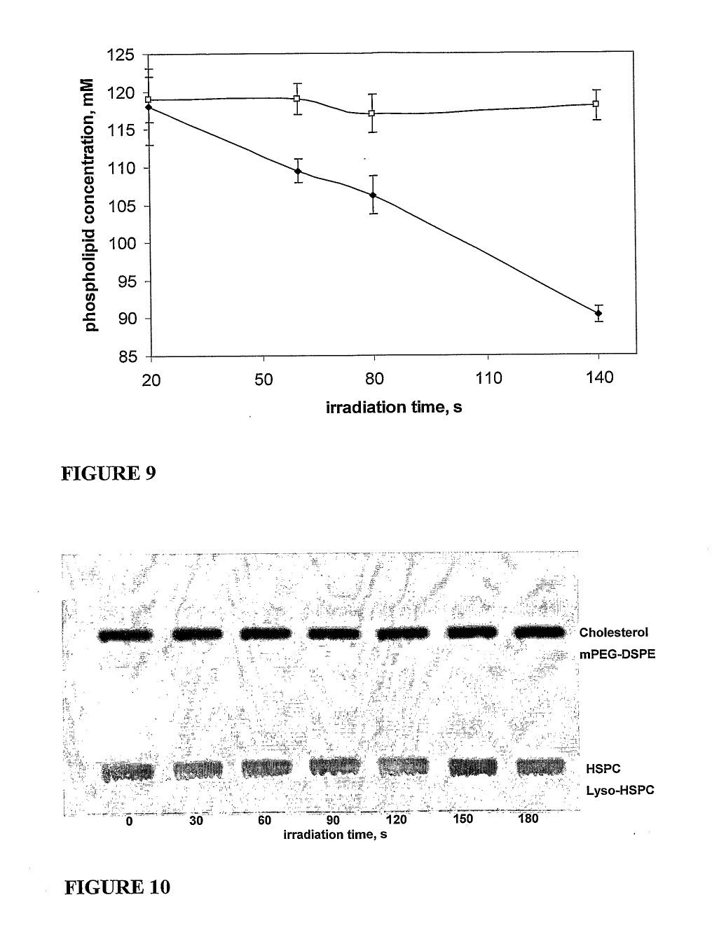

Determination of the amount of total phospholipid, and liposomal phospholipid (liposome peak in GPC) revealed that the amount of liposomal phospholipids decreased, while the total phospholipid remained unchanged with LFUS irradiation time (FIG. 9). This further supports the assumption presented herein that part of the liposomes are disassembled by LFUS. The fraction of disassembled liposomes after 140 s of LFUS irradiation was ˜23% of the irradiated liposomes; in agreement with reduction in DLS signal intensity and OD at 600 nm (FIG. 9). However, in the majority of liposomes, LFUS induces only transient porosity of the membrane, rather than complete liposome disassembly, and therefore the dominant effect is increased liposomal permeability, without altering liposome size distribution.

### Chemical Integrity of Ultrasonically Irradiated Phospholipids and Drugs

The chemical integrity of liposomal formulations, including irradiated drugs and lipids, was tested by HPLC for doxorubicin and MPS, by NMR for cisplatin, and by TLC for lipids.

SSL dispersions containing MPS, doxorubicin, and cisplatin were irradiated for periods of 30 to 180 s (20 kHz, 3.3 W/cm²) and then analyzed using as a reference non-irradiated liposomal dispersions. The HPLC chromatograms of LFUS-irradiated and non-irradiated drugs were identical, both for doxorubicin and MPS, as well as the NMR spectra for irradiated and non-irradiated cisplatin (data not shown), indicating, that LFUS, under the conditions used, does not induce any chemical changes in these three drugs.

Analysis of liposomal lipid extracts of irradiated and non-irradiated SSL by TLC show (FIG. 10) that no significant chemical changes occurred as a result of exposure to LFUS.

Cytotoxicity of a Drug Released from SSL by LFUS

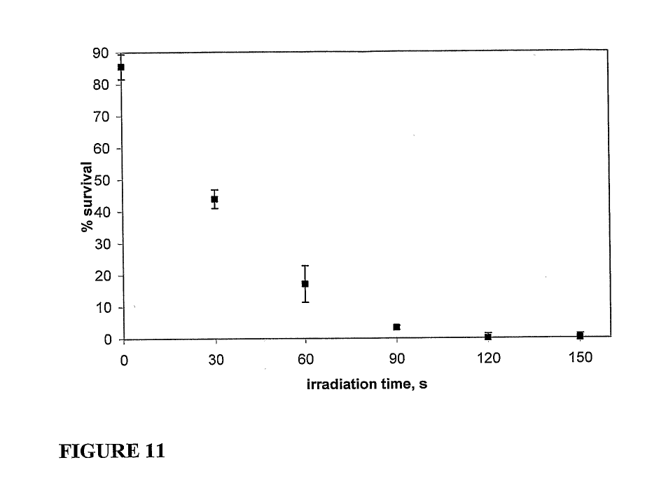

The cytotoxicity of an LFUS-released drug was tested by irradiating stealth cisplatin for different periods of time. Aliquots of these LFUS-irradiated dispersions were added to cultures of cisplatin-sensitive C26 murine colon adenocarcinoma cells for evaluation of drug cytotoxicity. As irradiation time increased, more cisplatin was released from the liposomes. The cytotoxicity was found to be proportional to the liposome irradiation time (FIG. 11) and similar to that of equal amounts of non-irradiated, free, cisplatin added to cells. Thus indicating that LFUS released liposomal cisplatin retained its biological activity.

### LFUS-Released Stealth Cisplatin in a Murine Lymphoma Model

Feasibility of LFUS to release drugs in vivo was tested in a murine lymphoma model. Stealth cisplatin was injected directly into the tumor to a depth of ˜2 cm. Then the tumor was irradiated by LFUS, and drug release was quantified as described above. Nearly 90% of stealth cisplatin exposed to LFUS was released, compared to less than 15% released from non-irradiated liposomes (data not shown), thus demonstrating feasibility of LFUS-induced liposomal drug release in vivo.

1.-68. (canceled)

69. A method for loading an agent into pre-formed liposomes comprising:
a. contacting pre-formed liposomes, being of a kind that can increase in permeability by an ultrasound irradiation, with said agent; and
b. subjecting the pre-formed liposomes to ultrasound irradiation, said irradiation having parameters being effective to increase permeability of said liposomes;
c. said contacting of the pre-formed liposomes with said agent is before or after said ultrasound irradiation.

70. The method of claim 69, wherein said ultrasound irradiation parameters comprise irradiation frequency, irradiation duration, irradiation intensity, number of irradiation sources per irradiation session, site of irradiation, number of irradiation sites, continuous or pulsed irradiation.

71. The method of claim 69, for loading of an agent into one or both of the liposome's leaflets.

72. The method of claim 69, wherein said agent is a hydrophobic or amphipathic molecule.

73. The method of claim 69, wherein said agent is a lipid.

74. The method of claim 71, wherein said agent is a lipid.

75. The method of claim 74, wherein said lipid is a liposome forming lipid or a non-liposome forming lipid.

76. The method of claim 75, wherein said lipid is a charged lipid, or a modified lipid.

77. The method of claim 69, wherein said pre-liposomes comprise one or more liposome forming lipids having a gel to liquid crystalline phase transition temperatures (Tm) equal or above 40° C.

78. The method of claim 77, wherein said liposomes comprise one or more liposome forming lipids in combination with cholesterol.

79. The method of claim 69, wherein said liposome forming lipid has a Tm equal or above 40° C. is selected from hydrogenated soy phosphatidylcholine (HSPC), Dipalmitoylphosphatidylcholine (DPPC), N-palmitoyl sphingomyelin, distearylphosphatidylcholine (DSPC), N-stearyl sphingomyelin, distearyolphosphatidylglycerol (DSPG), distearyphosphatidylserine (DSPS).

80. The method of claim 69, wherein said preformed liposomes have low permeability prior to said irradiation.

81. A method for reducing the amount of a substance in a fluid medium or tissue comprising:
a. contacting the fluid medium or tissue with pre-formed liposomes being of a kind that can increase in permeability by an ultrasound irradiation;
b. subjecting the pre-formed liposomes to ultrasound irradiation, said irradiation having parameters effective to increase permeability of said liposomes;
c. said contacting of the fluid medium or tissue with the pre-formed liposomes is before or after said ultrasound irradiation.

82. The method of claim 81, wherein said fluid medium is a biological fluid extracted from a subject's body, for reintroduction into the same or another subject after at least a portion of said substance is removed there from.

83. A method for reducing the level of a substance in a subject, the method comprises:
a. administering to said subject an amount of pre-formed liposomes, being of a kind that can increase in permeability by an ultrasound irradiation, in a manner permitting contact between said liposomes and said substance;
b. subjecting the pre-formed liposomes to ultrasound irradiation, and said ultrasound irradiation having parameters effective to increase permeability of said liposomes.

84. The method of claim 83, wherein said substance is in an area within said subject's body or in an extra-cellular fluid within the body.

85. The method of claim 83, wherein said irradiation takes place when said pre-formed liposomes are within said subject's body and said irradiation parameters are such that essentially no irreversible damage is caused to at least a portion of the subject's body as a result of said irradiation.

86. The method of claim 83, wherein said liposomes comprise one or more liposome forming lipids having a gel to liquid crystalline phase transition temperatures Tm equal or above 40° C.

87. The method of claim 83, wherein said liposomes comprise one or more liposome forming lipids in combination with cholesterol.

88. A method for the controlled quantum release from liposomes comprising an agent stably encapsulated therein and being of a kind that can increase in permeability by an ultrasound irradiation, the method comprises subjecting said liposomes to a series of two or more ultrasound irradiation sessions; each ultrasound irradiation having parameters effective to increase permeability of said liposomes thereby.
